(19) United States
(12) Patent Application Publication
(10) Pub. No.: US 2012/0295288 A1
(43) Pub. Date: Nov. 22, 2012

(54) SEROLOGICAL MARKER FOR DETECTING PANCREATIC CANCER AND A METHOD FOR USING THE SEROLOGICAL MARKER

(76) Inventors: Jau-Song Yu, Kwei-Shan (TW); Ya-Ting Chang, Taichung City (TW); Chih-Ching Wu, Pingtung City (TW); Yi-Ming Shyr, Taipei City (TW); Yu-Sun Chang, Linkou Shiang (TW)

(21) Appl. No.: 13/475,658

(22) Filed: May 18, 2012

(30) Foreign Application Priority Data
May 19, 2011 (TW) 100117502

(51) Int. Cl. G01N 33/574; H01J 49/26; G01N 27/62

(52) U.S. Cl. 435/7.94; 436/501; 435/7.1; 250/282

(57) ABSTRACT

UL16 binding protein 2 (ULBP2) is a protein overexpressed in pancreatic cancer tissues, and the ULBP2 levels are significantly higher in pancreatic cancer patients than those in healthy controls. This invention provides a method to detect pancreatic cancer using ULBP2 as a serological marker. The combination of ULBP2 and CA19-9 promotes the efficacy of pancreatic cancer detection. When measuring the blood ULBP2 levels in patients with other cancer types, including colorectal carcinoma, nasopharyngeal carcinoma and gastric cancer illustrates the blood ULBP2 levels are higher in patients with pancreatic cancer than other cancer types.

### FIELD OF THE INVENTION

Embodiments relate to a serological marker for detecting pancreatic cancer and a method for using the serological marker, especially to a serological marker with high sensitivity and specificity for detecting pancreatic cancer and a method for using the serological marker.

### BACKGROUND OF THE INVENTION

Pancreatic cancer is respectively ranked fourth and tenth among cancer-related mortality in United State and Taiwan and shows increased mortality these years. Because pancreas is anatomically located in a deeper site and the apparent symptoms of the pancreatic cancer is late onset, less than 8% of pancreatic cancer patients are diagnosed at the localized stage and can be surgically cured. More than 50% of diagnosed pancreatic cancer patients have exhibited distant metastasis, and the 5-year survival rate of which is less than 5%.

Current approaches for pancreatic cancer diagnosis are mainly based on imaging methods, such as abdominal sona or high-resolution computed tomography scan, and sometimes might combine with an invasive endoscopy or a magnetic resonance cholangiopancreatography to increase detection efficiency. However, the volume of pancreatic cancer in early stage is too small to be detected by the imaging method, which greatly increases the difficulty in detecting pancreatic cancer. With the development of molecular diagnosis and tumor biology, a serological marker, carbohydrate antigen 19-9 (CA 19-9), is widely applied in detecting pancreatic cancer. However, as current knowledge, CA 19-9 has few disadvantages in pancreatic cancer detection, such as the insufficient sensitivity and specificity and the unable detection of pancreatic cancer in early stage. Therefore, developing other serological markers to overcome above-mentioned disadvantages of CA19-9 and increase the detection efficiency will be an important issue to improve the management of pancreatic cancer.

### SUMMARY OF THE INVENTION

According to one aspect of an embodiment of the invention, a serological marker for detecting a pancreatic cancer with high sensitivity and specificity comprising at least an UL16 binding protein 2 (ULBP2) is provided. The ULBP2 is highly expression in pancreatic tumor tissues, and is significant increased in serum of a pancreatic cancer patient than in a normal healthy person. The ULBP2 is also significant increased in pancreatic cancer than in gastric cancer (GC), nasopharyngeal carcinoma cancer (NPC) and colorectal carcinoma cancer (CRC). Therefore, the ULBP2 is capable to be a serological marker for efficiently detecting pancreatic cancer by comparing its levels in the blood samples isolated from a patient and a normal healthy person.

According to another aspect of an embodiment of the invention, a bead-based immunoassay at least using an UL16 binding protein 2 (ULBP2) as a serological marker is used to detect pancreatic cancer and has exhibited an excellent sensitivity. The limitation of the bead-based immunoassay in detecting the pancreatic cancer is 3.91 pg/ml.

In a further embodiment of the invention, ULBP2 is combined with a carbohydrate antigen 19-9 (CA 19-9) to improve the detection efficiency. The ULBP2 also has ability to detect the pancreatic cancer at early stage and has more precise detection effect than CA19-9.

The above objects and advantages of the present invention will become more readily apparent to those ordinarily skilled in the art after reviewing the following detailed descriptions and accompanying drawings in which:

### BRIEF DESCRIPTIONS OF THE DRAWINGS

The patent or application file contains at least one drawing executed in color. Copies of this patent or patent application publication with color drawing(s) will be provided by the Office upon request and payment of the necessary fee.

FIG. 1 is a standard calibration curve of a serological marker for detecting pancreatic cancer by using a bead-based immunoassay.

FIG. 2 is a receiver operating characteristic curve of the ULBP2 and CA19-9 in detecting pancreatic cancer.

FIG. 3A shows the detection efficiency of ULBP2, CA19-9 and ULBP2 combined with CA19-9 in detecting the pancreatic cancer of T1/T2 stage of the TNM classification.

FIG. 3B shows the detection efficiency of ULBP2, CA19-9 and ULBP2 combined with CA19-9 in detecting the pancreatic cancer of N0 stage of the TNM classification.

FIG. 3C shows the detection efficiency of ULBP2, CA19-9 and ULBP2 combined with CA19-9 in detecting the pancreatic cancer of I-II stage of the overall stage.

FIG. 4 shows the immunohistochemistry assay of the cancer tissue biopsies from 67 pancreatic cancer patients shows that ULBP2 staining is positive in all biopsies (100%), and the expression of ULBP2 is more significant in the cancer tissue than in the non-cancerous tissues.

### DETAILED DESCRIPTION OF THE PREFERRED EMBODIMENT

### Embodiment 1

### Screening and Selecting a Serological Marker for Detecting the Pancreatic Cancer

Proteins secreted from two pancreatic cancer cell lines PANC-1 and MIA PaCa-2, which were collected by incubating the cultured cells in serum-free medium for 24 hr (this medium is thereafter defined as conditioned medium), are systematically identified by one-dimensional SDS-PAGE in conjunction with nano-LC-MS/MS (the GeLC-MS/MS approach). This method identified a total of 1812 non-redundant proteins from the conditioned medium of the two cell lines. The transcriptional expression of each identified protein in the pancreatic cancer tissues was further analyzed according to a public domain transcriptomic information of the pancreatic cancer tissues (National Center for Biotechnology Information (NCBI) Gene Expression Omnibus). In this transcriptome dataset, pancreatic ductal cells respectively isolated from 25 healthy donors and 24 pancreatic cancer patients are subjected to an array-based analysis to identify the genes whose message RNA (mRNA) levels are higher expressed in the pancreatic cancer patients than those in the healthy donors. By integrating this transcriptome dataset and the secreted protein database of the two pancreatic cancer cell lines generated as described above, 30 pancreatic cancer cell secreted proteins exhibited at least two-fold higher mRNA expression levels in the pancreatic ductal cells from cancer patients than from the healthy donors. Eleven out of the 30 proteins had been reported to be over-expressed in the tissue biopsy of pancreatic cancer by previous studies. Among the other 19 proteins without any references related to pancreatic cancer, UL16 binding protein 2 (ULBP2) is selected as a serological candidate marker for detecting pancreatic cancer in the present invention.

The ULBP2 in the present embodiment has an amino acid sequence (SEQ ID
NO: 1) shown as following:
Met Ala Ala Ala Ala Ala Thr Lys Ile Leu Leu Cys Leu Pro Leu Leu Leu Leu Leu
Ser Gly Trp Ser Arg Ala Gly Arg Ala Asp Pro His Ser Leu Cys Tyr Asp Ile Thr Val
Ile Pro Lys Phe Arg Pro Gly Pro Arg Trp Cys Ala Val Gln Gly Gln Val Asp Glu
Lys Thr Phe Leu His Tyr Asp Cys Gly Asn Lys Thr Val Thr Pro Val Ser Pro Leu
Gly Lys Lys Leu Asn Val Thr Thr Ala Trp Lys Ala Gln Asn Pro Val Leu Arg Glu
Val Val Asp Ile Leu Thr Glu Gln Leu Arg Asp Ile Gln Leu Glu Asn Tyr Thr Pro
Lys Glu Pro Leu Thr Leu Gln Ala Arg Met Ser Cys Glu Gln Lys Ala Glu Gly His
Ser Ser Gly Ser Trp Gln Phe Ser Phe Asp Gly Gln Ile Phe Leu Leu Phe Asp Ser
Glu Lys Arg Met Trp Thr Thr Val His Pro Gly Ala Arg Lys Met Lys Glu Lys Trp
Glu Asn Asp Lys Val Val Ala Met Ser Phe His Tyr Phe Ser Met Gly Asp Cys Ile
Gly Trp Leu Glu Asp Phe Leu Met Gly Met Asp Ser Thr Leu Glu Pro Ser Ala Gly
Ala Pro Leu Ala Met Ser Ser Gly Thr Thr Gln Leu Arg Ala Thr Ala Thr Thr Leu
Ile Leu Cys Cys Leu Leu Ile Ile Leu Pro Cys

One ordinary skill in the art understands that any amino acid replacement by an amino acid with similar characteristic will cause a little variation in the original SEQ ID NO:1. However, a sequence has similarity more than 95% to SEQ ID NO:1 is considered as a serological marker for detecting pancreatic cancer that can be used in the embodiment.

### Embodiment 2

### The Expression of the Serological Marker ULBP2 in Pancreatic Cancer Tissues

Immunohistochemistry assay: In the embodiment, a goat anti-ULBP2 antibody is applied. A tissue biopsy is isolated and heated in a 0.01M citric acid buffer (pH 6.0). A blocking buffer is added and reacted at room temperature for 5 minutes. The tissue biopsy is reacted with the anti-ULBP2 antibody (1:20 dilution) at 4° C. for 16 hours. Then, the tissue biopsy is stained with the N-Histofine® (Nichirei, Japan) at room temperature and followed by treatment with substrate DAB chromogen (Novocastra/Leica Microsystems, IL, USA). The tissue biopsy is also counterstained with hematoxylin. The expression level of target proteins was evaluated according to the simplified H score system, which is based on the intensity of cell staining [3 (strong), 2 (moderate), 1 (weak), or 0 (no cell staining)] and the percentage of cell staining [3 (≧90%), 2 (50-89%), 1 (10-49%), or 0 (0-9%)]. The two scores were multiplied by each other and then divided by 3 to get the final score. Positive staining was defined as a final score≧0.67.

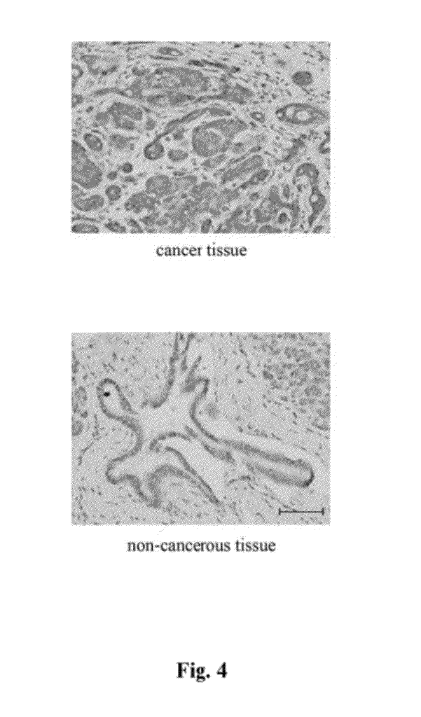

With reference to the FIG. 4, the immunohistochemistry assay of the cancer tissue biopsies from 67 pancreatic cancer patients shows that ULBP2 staining is positive in all biopsies (100%). Additionally, the expression of ULBP2 is more significant in the cancer tissue than in the adjacent non-cancerous tissues (the intensity of brown color indicates the expression level of the ULBP2). The mean expression level of the ULBP2 in the cancer tissue and in the adjacent non-cancerous tissue is score of 2.71±0.49 and 1.89±0.74, respectively. Moreover, with reference to Table 1, the expression of ULBP2 of the embodiment is not influenced by the clinically pathological symptoms such as the gender, age, histological grade, overall cancer status or TMN classification. The detecting result of the ULBP2 has highly consistence in patients with different clinically pathological symptoms.

TABLE 1
Correlation between clinicopathological features and ULBP2 expression
in 67 pancreatic cancer patients

| | | IHC score | |
|---|---|---|---|
| Characteristics | Patient No. | (Mean ± SD)^{a} | p-value |
| Gender | | | |
| Male | 44 | 2.64 ± 0.54 | 0.180^{b} |
| Female | 23 | 2.83 ± 0.36 | |
| Age (years) | | | |
| <64^{c} | 33 | 2.61 ± 0.55 | 0.089^{b} |
| ≧64 | 34 | 2.80 ± 0.41 | |
| Histological grade^{d} | | | |
| Well differentiation | 23 | 2.71 ± 0.59 | 0.746^{e} |
| Moderate differentiation | 31 | 2.68 ± 0.44 | |
| Poor differentiation | 10 | 2.70 ± 0.48 | |
| Overall stage | | | |
| stage I | 9 | 2.52 ± 0.82 | 0.593^{e} |
| stage II | 56 | 2.73 ± 0.43 | |
| stage IV | 2 | 3.00 ± 0.00 | |
| Tumor-node-metastasis | | | |
| (TNM)-T classification | | | |
| TNM-T2 | 9 | 2.52 ± 0.82 | 0.653^{b} |
| TNM-T3 | 58 | 2.74 ± 0.42 | |
| TNM-N classification | | | |
| TNM-N0 | 25 | 2.69 ± 0.59 | 0.806^{b} |
| TNM-N1 | 42 | 2.72 ± 0.43 | |
| TNM-M classification | | | |
| No metastasis | 65 | 2.70 ± 0.50 | 0.479^{b} |
| Distant metastasis | 2 | 3.00 ± 0.00 | |

^{a}Intensity and percentage scores of cell staining were multiplied by each other and then divided by 3 to get the IHC scores.
^{b}By Wilcoxon test.
^{c}Median.
^{d}Histological grade information not available in 3 patients.
^{e}By Kruskal-Wallis test.

### Embodiment 3

### The Levels of the Pancreatic Cancer Serological Marker ULBP2 in Serum Sample of Pancreatic Cancer Patients

A bead-based immunoassay is used to detect the ULBP2 level in a serum sample. An ULBP2 antibody, used as a capture antibody, is pre-coupled to COOH beads using the Bio-Plex Amine Couplin Kit (Bio-Rad). A biotin-conjugated anti-ULBP2 antibody is used as a detection antibody. The bead with the capture antibody is added in a filter-bottom 96-well microplate (Millipore). Then the serum sample solution or standard solution containing ULBP2 protein at various concentrations (3.91˜3.2×10⁴ pg/mL) is added into the well to react in dark at room temperature for 1 hour. After washing the serum sample solution or the standard solution, the detection antibody is added into each well and reacted in dark at room temperature for 1 hour. After washing out the detection antibody, the phycoerythrin-conjugated streptavidin solution is added for 10 minutes to allow the binding between streptavidin and biotin. Unbound streptavidin is removed by a wash step. The ULBP2 level in the serum sample is then calculated by the fluorescent strength of the phycoerythrin based on the fluorescent strength of the standard calibration curve.

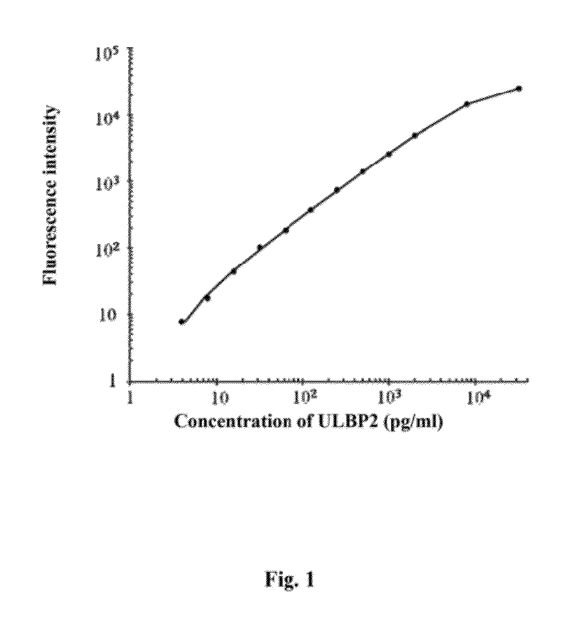

With reference to FIG. 1, the ULBP2 in the serum sample is very easy to be detected using the bead-based immunoassay. The ULBP2 concentration ranged from 4.3 pg/mL to 31.9 ng/mL in the embodiment is precisely detected, which is not stably and accurately performed in a sandwich ELISA assay. The ULBP2 level is significantly increased in the cancer serum samples (200.2±168.6 pg/ml) than in the healthy control samples (51.4±64.6 pg/ml). When a cutoff value of 60 pg/mL for ULBP2 is chosen, the sensitivity and specificity values for cancer detection is 83.8% and 73.9%, respectively. These findings indicate that ULBP2 is a novel serum marker for pancreatic cancer detection. However, the serum ULBP2 levels are not statistically correlated with age, gender, histological grade, tumor overall stage, and TNM classification of pancreatic cancers in this case control study (Table 2).

TABLE 2
Correlation of serum ULBP2 and CA19-9 with clinicopathologic
characteristics in 154 pancreatic cancer patients.

| | | ULBP2 | | CA19-9 | |
|---|---|---|---|---|---|
| | | (pg/mL) | | (U/mL) | |
| Characteristics | No. | Mean ± SD | p-value | Mean ± SD | p-value |
| Gender | | | | | |
| Male | 111 | 199.5 ± 168.4 | | 64.7 ± 24.6 | |
| Female | 43 | 202.0 ± 170.8 | | 60.1 ± 23.7 | |
| Age (years) | | | | | |
| <70^{b} | 75 | 200.4 ± 182.9 | | 60.2 ± 26.0 | |
| ≧70 | 79 | 199.9 ± 154.9 | | 66.4 ± 12.5 | |
| Histological | | | | | |
| grade^{c} | | | | | |
| Well | 12 | 148.8 ± 101.0 | 0.377^{d} | 70.0 ± 13.7 | 0.553^{d} |
| differentiation | | | | | |
| Moderate | 102 | 198.7 ± 173.1 | | 59.8 ± 26.0 | |
| differentiation | | | | | |
| Poor | 14 | 134.4 ± 99.9 | | 58.7 ± 25.2 | |
| differentiation | | | | | |
| Overall stage^{c} | | | | | |
| Stage I-II | 106 | 181.2 ± 158.8 | | 60.7 ± 24.4 | |
| Stage III-IV | 22 | 215.3 ± 178.5 | | 60.4 ± 28.9 | |
| Tumor-node- | | | | | |
| metastasis | | | | | |
| (TNM)-T | | | | | |
| classification^{c} | | | | | |
| TNM-T1 | 7 | 251.0 ± 150.0 | 0.418^{d} | 62.1 ± 20.5 | 0.536^{d} |
| TNM-T2 | 27 | 194.0 ± 192.9 | | 54.8 ± 28.4 | |
| TNM-T3 | 77 | 175.1 ± 150.5 | | 61.8 ± 24.0 | |
| TNM-T4 | 17 | 204.0 ± 171.1 | | 63.9 ± 26.8 | |
| TNM-N | | | | | |
| classification^{c} | | | | | |
| TNM-N0 | 57 | 191.6 ± 155.2 | | 62.1 ± 25.5 | |
| TNM-N1 | 71 | 183.4 ± 168.5 | | 59.4 ± 24.9 | |
| TNM-M | | | | | |
| classification^{c} | | | | | |
| No metastasis | 125 | 190.5 ± 162.4 | | 60.9 ± 25.2 | |
| Distant | 3 | 40.8 ± 27.7 | | 48.5 ± 22.1 | |
| metastasis | | | | | |

^{a}By Wilcoxon test.
^{b}Median.
^{c}Information of histological grade, overall stage and TNM stage not available in 26 patients.
^{d}By Kruskal-Wallis test.

The performance of the currently used pancreatic cancer marker CA 19-9 and the pancreatic cancer serological marker ULBP2 in 154 pancreatic cancer patients is compared to evaluate their detection efficacy. Both CA19-9 and ULBP2 show elevated serum levels in the pancreatic cancer patients than in the healthy controls and are not influenced by the clinicopathological characteristics. At 40 U/mL of CA 19-9, a cutoff value currently applied for pancreatic cancer screening in clinics, the sensitivity and specificity values is 84.4% and 74.6%, respectively. Noteworthily, upon selection of a cutoff value of 60 pg/mL for ULBP2, 21 of 24 pancreatic cancer patients with CA 19-9 levels<40 U/mL could be discriminated form healthy individuals based on ULBP2 levels>60 pg/mL. In addition, 24 of 36 healthy individuals with CA 19-9 levels>40 U/mL could be further distinguished form the patients based on ULBP2 levels<60 pg/mL. The combined usage of ULBP2 and CA19-9 has a great benefit to pancreatic cancer detection by using CA19-9 alone (shown in Table 3).

TABLE 3
The efficacy of ULBP2 and CA 19-9 for detecting pancreatic cancers.

| | | ULBP2 | ULBP2 |
|---|---|---|---|
| | Total Sample | (>60 pg/mL) | (<60 pg/mL) |
| Cancer patients (n = 154) | | | |
| CA19-9 (<40 U/mL) | 24 | 21 | 3 |
| CA19-9 (>40 U/mL) | 130 | 108 | 22 |
| Healthy controls (n = 142) | | | |
| CA19-9 (<40 U/mL) | 118 | 24 | 82 |
| CA19-9 (>40 U/mL) | 36 | 12 | 24 |

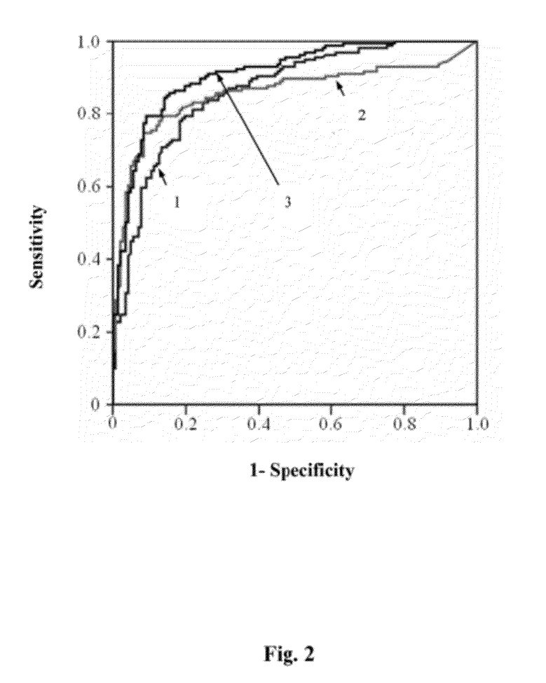

With reference to FIG. 2, the abilities of ULBP2 and CA 19-9 as detection markers are further tested by receiver operator characteristic (ROC) curve analysis and area under the ROC curve (AUC). The analysis demonstrated that ULBP2 (line 1) [AUC=0.862, 95% confidence interval (CI), 0.821-0.904] is slightly better than CA 19-9 (line 2) (AUC=0.856, 95% CI, 0.809-0.902) as a screening marker. Most importantly, the combination of ULBP2 and CA 19-9 (line 3) using the logistic regression model shows a higher diagnostic capacity than either marker alone (AUC=0.910, 95% CI, 0.877-0.943). These results collectively reveal that ULBP2 is a useful serum marker for pancreatic cancer, especially when used together with CA 19-9.

### Embodiment 5

### The Ability of the Pancreatic Cancer Serological Marker ULBP2 for Early Detection of Pancreatic Cancer

142 healthy specimens and 154 pancreatic cancer patients are enrolled to evaluate the ability of ULBP2 for early detection of pancreatic cancers. The ULBP2 level in serum samples of the healthy controls is 51.4±64.6 pg/mL that is less than that in the pancreatic cancer patients at any stage (TNM classification-T1/T2, TNM classification-N0 and overall Stage I-II is 205.7±184.3 pg/mL, 191.6±155.2 and 181.2±158.8 pg/mL, respectively, p<0.0001). The results are similar to the CA19-9 and indicate the pancreatic cancer serological marker ULBP2 is able to use for early detection of pancreatic cancer.

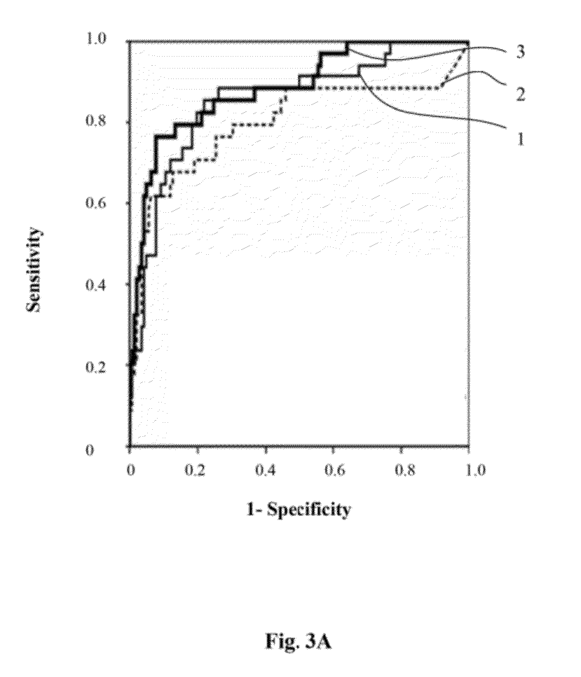

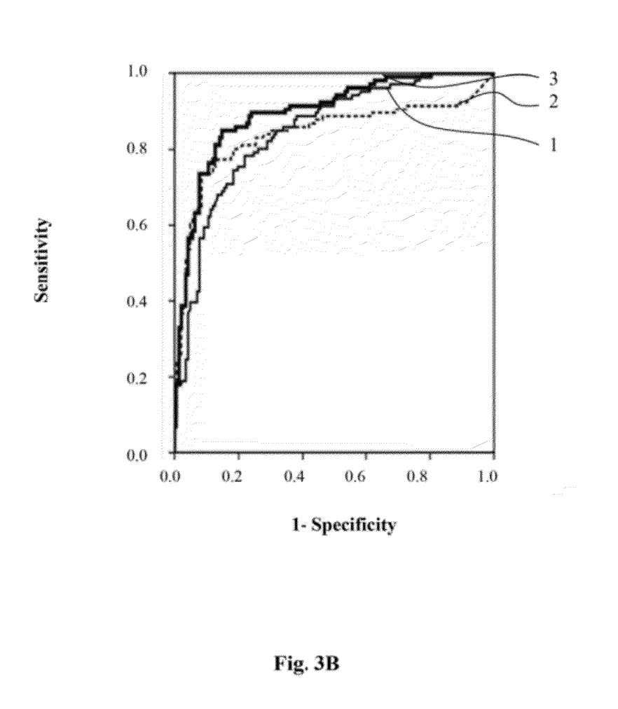

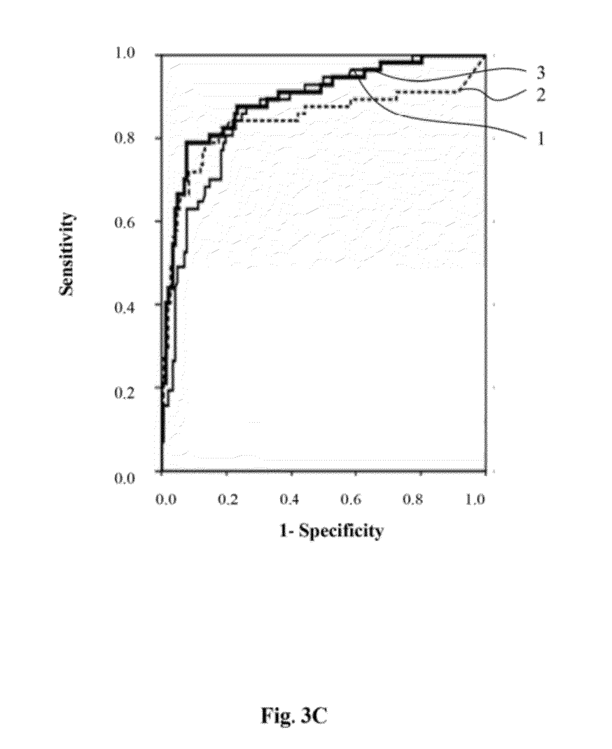

With reference to FIGS. 3A to 3C, the ROC analysis shows that ULBP2 has better performance in early detection of pancreatic cancer than CA19-9. Furthermore, the detection efficiency is improved by combining ULBP2 and CA19-9.

TABLE 4
The ability of ULBP2 and CA 19-9 for early detection of pancreatic
cancers

| | | | | | ULBP2 conbined | |
|---|---|---|---|---|---|---|
| | ULBP2 | | CA 19-9 | | with CA 19-9 | |
| | AUC | 95% CI | AUC | 95% CI | AUC | 95% CI |
| TNM | 0.854 | 0.778~0.930 | 0.796 | 0.690~0.901 | 0.883 | 0.816~0.949 |
| classifi- | | | | | | |
| cation- | | | | | | |
| T1/T2 | | | | | | |
| TNM | 0.866 | 0.811~0.920 | 0.841 | 0.764~0.917 | 0.893 | 0.841~0.946 |
| classifi- | | | | | | |
| cation- | | | | | | |
| N0 | | | | | | |
| Overall | 0.846 | 0.798~0.895 | 0.839 | 0.782~0.896 | 0.897 | 0.856~0.937 |
| Stage | | | | | | |
| I-II | | | | | | |

### Embodiment 6

### The Specificity of the Pancreatic Cancer Serological Marker ULBP2

The specimens obtained from gastric cancer (GC), nasopharyngeal carcinoma cancer (NPC) and colorectal carcinoma cancer (CRC) patients are applied to evaluate the specificity of the pancreatic cancer serological marker ULBP2. The ULBP2 levels in serum samples from the NPC and CRC, and plasma samples from the GC are detected. As shown in Table 5, compared with the healthy controls (51.4+64.6 pg/mL for serum ULBP2), the serum ULBP2 levels are slightly higher in patients suffered from NPC (N=28, 65.5±74.3 pg/mL, p=0.122) or CRC (N=29, 70.6±73.8 pg/mL, p=0.038). However, the serum ULBP2 levels are significantly elevated in pancreatic cancer compared to that in CRC (200.2±168.6 versus 70.6±73.8 pg/mL, p<0.0001) and NPC (200.2±168.6 versus 65.5±74.3 pg/mL, p<0.0001). The results illustrate that ULBP2 represents a relative specific marker for pancreatic cancer, particularly that its level do not alter or only marginally elevated in the other two gastrointestinal cancers, CRC and GC.

TABLE 5
The detection efficiency of the pancreatic cancer serological marker
ULBP2 in different cancers.

| | | ULBP2 in serum | ULBP2 in plasma |
|---|---|---|---|
| | Sample No. | (pg/mL) | (pg/mL) |
| Pancreatic | 154 | 200.2 ± 168.6 | — |
| cancer | | | |
| GC | 30 | — | 78.1 ± 79.7 |
| NPC | 28 | 65.5 ± 74.4 | — |
| CRC | 29 | 70.6 ± 73.8 | — |
| Control 1 | 142 | 51.4 ± 64.6 | — |
| Control 2 | 25 | — | 86.1 ± 101.2 |

Accordingly, the above-mentioned embodiments illustrate the serological marker ULBP2 is significant increased in the serum of a pancreatic cancer patient and is not correlative with the clinicophathological characteristics. The detection sensitivity of the serological marker ULBP2 is sharply improved to 3.91 pg/mL in the serum sample. The serological marker ULBP2 has ability to detect the pancreatic cancer at early stage. The ULBP2 is combined with the CA19-9 to promote the efficiency and increase the specificity in pancreatic cancer detection, also in the early stage cancer detection. Therefore, the serological marker ULBP2 actually has capability to detect the pancreatic cancer in the early stage and strength the efficiency of the clinical diagnosis.

What is claimed is:

1. A method for detecting pancreatic cancer, comprising steps of
sampling: getting a blood specimen from a testee,
detecting: at least detecting the level of an UL16 binding protein 2 (ULBP2) in the blood specimen,
calculating: calculating a concentration of the ULBP2 by using a stand calibration curve and comparing the concentration of the ULBP2 with a control concentration of the ULBP2 of a blood specimen from a healthy person.

2. The method as claimed in claim 1, wherein the ULBP2 having an amino acid sequence of SEQ ID No. 1.

3. The method as claimed in claim 1, wherein the ULBP2 having similarity of 95% with an amino acid sequence of SEQ ID No. 1.

4. The method as claimed in claim 1, wherein the step of detecting further comprising at least additional serological marker of detecting a pancreatic cancer

5. The method as claimed in claim 1, wherein the additional serological marker is carbohydrate antigen 19-9 (CA 19-9).

6. The method as claimed in claim 1, wherein the blood specimen is a whole blood, a serum or a plasma blood specimen.

7. The method as claimed in claim 1, wherein the serological marker is applied in a bead-based immunoassay, a sandwich enzyme-linked immunosorbent assay (ELISA), a mass spectrometry-based assay or a mass spectrometry-based immunoassay.

8. The method as claimed in claim 2, wherein the serological marker is applied in a bead-based immunoassay, a sandwich enzyme-linked immunosorbent assay (ELISA), a mass spectrometry-based assay or a mass spectrometry-based immunoassay.

9. The method as claimed in claim 3, wherein the serological marker is applied in a bead-based immunoassay, a sandwich enzyme-linked immunosorbent assay (ELISA), a mass spectrometry-based assay or a mass spectrometry-based immunoassay.

10. The method as claimed in claim 4, wherein the serological marker is applied in a bead-based immunoassay, a sandwich enzyme-linked immunosorbent assay (ELISA), a mass spectrometry-based assay or a mass spectrometry-based immunoassay.

11. The method as claimed in claim 5, wherein the serological marker is applied in a bead-based immunoassay, a sandwich enzyme-linked immunosorbent assay (ELISA), a mass spectrometry-based assay or a mass spectrometry-based immunoassay.


SEQUENCE LISTING (1 sequence)

SEQ ID NO: 1 - protein, 240 aa, Homo sapiens
MAAAAATKILLCLPLLLLLSGWSRAGRADPHSLCYDITVIPKFRPGPRWCAVQGQVDEKTFLHYDCGNKTVTPVSPLGKKLNVTTAWKAQNPVLREVVDILTEQLRDIQLENYTPKEPLTLQARMSCEQKAEGHSSGSWQFSFDGQIFLLFDSEKRMWTTVHPGARKMKEKWENDKVVAMSFHYFSMGDCIGWLEDFLMGMDSTLEPSAGAPLAMSSGTTQLRATATTLILCCLLIILPC